(19) United States
(12) Patent Application Publication
(10) Pub. No.: US 2007/0299537 A1
(43) Pub. Date: Dec. 27, 2007

(54) Cell Culture Method, Three-Dimensional Cell Culture Method, Three-Dimensional Tissue, Artificial Organ and Tissue Transplantation Method

(76) Inventors: Ryo Sudo, Tokyo (JP); Kazuo Tanishita, Maguro-Ku (JP); Mariko Ikeda, Chiba (JP); Toshihiro Mitaka, Sapporo (JP)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/579,294

(22) PCT Filed: Nov. 12, 2004
(86) PCT No.: PCT/JP04/17227

§ 371 Date: May 2, 2007

(30) Foreign Application Priority Data
Nov. 14, 2003 (JP) 2003-385677

(51) Int. Cl. A61F 2/02; A01N 1/00; C12N 5/08

(52) U.S. Cl. 623/23.72; 435/1.1; 435/395

(57) ABSTRACT

Cultured cells flat-cultured on a permeable sheet is stacked on other flat-cultured cells together with the permeable sheet to construct a three-dimensional tissue. The three-dimensional tissue is transplanted into a living body. Alternatively, the three-dimensional tissue is stacked up in an artificial organ device to construct a stacked-up, three-dimensional bioartificial organ module. Colony form of the cultured cell can be controlled by using a microporous sheet as the permeable sheet and controlling positions of pores in the microporous sheet to design the form of the artificial organ.

### CROSS-REFERENCE TO RELATED APPLICATIONS

This application claims priority to a Japanese patent application No. 2003-385677 filed on Nov. 14, 2003, the disclosure of which incorporated herein by reference.

### FIELD OF THE INVENTION

The present invention relates to a cell culture method to construct three-dimensional tissues, a three-dimensional tissue constructed from the cultured cells, an artificial organ, and a tissue transplantation method.

### BACKGROUND OF THE INVENTION

In the field of transplantation therapy, therapeutic methods have been studied to culture cells in vitro, from which a tissue or organ of living body is reconstructed under culture condition and transplanted into a patient as an artificial organ.

Among tissues and organs, the liver is a central organ of metabolisms with various complex functions such as digestion and detoxification, exerting over 500 known metabolic reactions. Insufficiency in liver function could become lethal. Due to functional complexity of the liver, substitution of a liver by a fully-artificial device is extremely difficult, and the use of hepatic cells from a living body is believed to be the only way for a long-term substitution. Current method for radical treatment of patients with severe hepatic failure is a liver transplantation from a living donor, but the number of organ donors are limited, and developments of a method for organ reconstitution under culture condition and/or an artificial organ is highly anticipated for the liver among others.

Currently the developments are under way for hybrid-type artificial livers using hepatic cells cultured in vitro. Mostly used are the methods where hepatic cells are filled in a reactor of hollow fibers and materials are exchanged through semi-permeable membranes. Other types of various artificial livers developed so far include floating-cell type, stacked-up type, collagen-sandwiched type, microcarrier-attached type, and microcapsule-encapsulated type (see Matsushita, Michiaki, et al. 1998, “Bioartificial liver.” The Tissue Culture Engineering 14(5), 188-192, for example).

Constructions of such hybrid-type artificial livers require hepatic cells cultured at high density in a three-dimensional form. Hepatic cells in a conventional monolayer culture lose function within a few days and die in 7 to 10 days, and constructing a usable artificial liver from them is difficult. Therefore, culture methods utilizing spheroids or temperature-responsive culture dish have been developed.

In the spheroid culture method, hepatocyte spheroids partially attached to the bottom of a culture dish are obtained by seeding isolated hepatocytes on the dish that has been treated electrically or by macromolecules. In this method, synthesis of albumins from the hepatocyte spheroids and the hepatocytes can be observed for longer than one month.

A hybrid-type artificial liver module utilizing the spheroid culture method has been also developed. By culturing hepatocytes in the pores of polyurethane foam (PUF), a biocompatible macromolecular material, approximately two hundred of the hepatocytes aggregate to spontaneously form a spherical tissue mass (spheroid) with a diameter around 150 μm. Taking advantage of this fact, an artificial liver module has been developed where a large number of tubules are bored in a cylindrical PUF block for liquid flow and many hepatocyte spheroids are formed in the pores between the tubules in the PUF.

Alternatively, in the method of hepatocyte culture utilizing the temperature-responsive culture dish, a culture dish whose surface is grafted by poly-N-isopropyl acrylamide (PNPAAm), a temperature-responsive macromolecule, is used. While bottom surface of the dish is hydrophobic at culturing temperature (37° C.) so that the cells remain attached to the dish, the surface becomes highly hydrophilic at lower temperature (below 32° C.) and the cells detach from the dish surface spontaneously without losing their structure and function. When cells are cultured at high density in this culture method, a cellular layer comprising the cells and an extracellular matrix (ECM) can be obtained. Attempts have been made to reconstruct a liver by stacking up this monolayer of the cells.

However, no successful organization of organs such as liver under culture condition is reported so far.

Thus, the objective of the present invention is to provide a cell culture method to construct a three-dimensional tissue, a three-dimensional tissue constructed from the cultured cells, an artificial organ, and a tissue transplantation method.

### BRIEF SUMMARY OF THE INVENTION

Although liver is known to be capable of regenerating actively, a hepatocyte loses its function rapidly once isolated ex vivo. Then, the inventors successfully enabled a long-term culture of hepatocytes by way of using a progenitor of the hepatocyte called “small hepatocyte”. Small hepatocytes cultured on a collagen-coated microporous polycarbonate sheet can attach to the sheet and proliferate. As shown in FIG. 1 C1 to 5, when multiple sheets with the cells having been attached and cultured for 30 days were stacked up, they adhered to each other by the cells of the upper and lower layers adhered to each other. Observation of the fine structure of their vertical section by a transmission electron microscope revealed that structures similar to bile canaliculi had been formed between them, and thus the present invention was completed.

The three-dimensional cell culture method of the present invention includes constructing a three-dimensional tissue comprising multiple layers by stacking cells flat-cultured on a permeable sheet on other flat-cultured cells together with the permeable sheet. As used herein, the “three-dimensional tissue” is a steric cluster of cells, in which the cells are not only sterically clustered, but also interact to each other and exert certain function(s) by their association. The function can preferably be, but not limited to, the function of the original tissue from which the cells were originated, and a new function can be attained by differentiation of the cells in the cases where the cells are multipotent cells, such as stem cells. Alternatively, a function different from the function of the original tissue can be attained by transdifferentiation of the cells.

In accordance with the culture method of the present invention, the cultured cells can be originated from any one of a solid organ, an epithelial tissue, or a muscular tissue, preferably from a liver, and most preferably from a small hepatocyte. As used herein, the “solid organ” is the organ with solid content, such as liver, kidney, pancreas, and spleen. As used herein, the “epithelial tissue” is the tissue which covers the surfaces of a body, a lumen (such as digestive tract, respiratory tract, urinary tract, genital tract, and blood vessel), or a cavity (such as pericardial cavity, pericardial cavity, and peritoneal cavity), and includes the epithelia in the narrow sense, endothelium, and mesothelium, such as gastrointestinal epithelium, corneal epithelium, vascular endothelium, and pleural mesothelium. As used herein, the “muscular tissue” is the cardiac muscle, a smooth muscle, or a skeletal muscle. Additionally, the cells can be originated from multiple origins, as exemplified in the cases where the tissue is the epithelium in a solid organ (such as the vascular endothelium in a liver).

In the three-dimensional tissue constructed by the three-dimensional tissue culture method of the present invention using a cell originated from a liver, a bile canaliculus is preferably formed.

The three-dimensional tissue of the present invention is constructed by stacking cells flat-cultured on a permeable sheet on other flat-cultured cells together with the permeable sheet.

The cell to be used to construct the three-dimensional tissue can be originated from any one of a solid organ, an epithelial tissue, or a muscular tissue, preferably from a liver, and most preferably, a small hepatocyte.

In the three-dimensional tissue constructed by using a cell originated from the liver, a bile canaliculus is preferably formed.

The artificial organ of the present invention is constructed from the three-dimensional tissue described above. As used herein, the “artificial organ” includes the whole tissues of an artificially constructed living body, and includes, but not limited to, the organ in the narrow sense, as well as a sterical cluster of cells being organized and functioning, such as epithelia, muscles, and nerves.

The cell culture method of the present invention is a cell culture method of flat-culturing cells on a permeable sheet includes defining the colony form of the cultured cells by controlling the position of a pore in the permeable sheet.

The cultured cells cultured by the cell culture method of the present invention can be stacked on other flat-cultured cells together with the permeable sheet to construct a three-dimensional tissue.

In accordance with the tissue transplantation method of the present invention, the three-dimensional tissue described above is transplanted into a living body of a non-human vertebrate. This tissue transplantation method is applicable to humans as well.

### BRIEF DESCRIPTION OF THE DRAWINGS

FIG. 1 shows schematic diagrams of the method to construct a three-dimensional tissue in accordance with one embodiment of the three-dimensional culture method of the present invention. The cellular sheets shortly after the cells were seeded (A) and after cells had been cultured (B) are shown. (C)1 to 5 illustrates the method of the stacking.

FIG. 2 shows schematic diagrams of a stacked-up, three-dimensional bioartificial liver module (A), and a stacked-up artificial liver device constructed from the liver module shown in A (B), in accordance with one embodiment of the artificial organ of the present invention.

FIG. 3 shows transmission electron micrographs of vertical sections of a three-dimensional tissue constructed in accordance with one embodiment of the three-dimensional culture method of the present invention. The white arrow in (A) indicates a desmosome, and the white arrow in (B) indicates a microvillus in the lumen of a bile canaliculus.

FIG. 4 shows a photograph of bile canaliculi which were formed in the three-dimensional tissue constructed and stained by fluorescein in accordance with one embodiment of the three-dimensional culture method of the present invention. Bright portions indicate fluorescent signals.

FIG. 5 shows a graph indicating time courses of the amount of albumins secreted into culture media in accordance with one embodiment of the three-dimensional culture method of the present invention.

FIG. 6 shows a photograph indicating that the colony form is defined by the positions of pores (circular dark portions) in accordance with one embodiment of the three-dimensional culture method of the present invention.

FIG. 7 shows the results from an examination of the expression of hepatic differentiation markers in the cells of a three-dimensional tissue formed by one embodiment of the three-dimensional culture method of the present invention. “PH” indicates RNA extracted from mature hepatocytes isolated from a rat, and “S5 or S10” indicates RNA extracted five or ten days after the three-dimensional tissue construction from the respective three-dimensional tissues.

Reference letters used in the drawings are as follows:

1 Cell;
2 Permeable sheet;
3 Cell culture dish.

### DETAILED DESCRIPTION OF THE PREFERRED EMBODIMENTS

The followings are the detailed description of the cell culture method, the three-dimensional cell culture method, the three-dimensional tissue formed by using the culture method, the artificial organ constructed from the formed three-dimensional tissues, and the tissue transplantation method of the three-dimensional tissues, in accordance with the present invention, with references to the drawings.

The objective, the specifications, the advantages and the ideas of the present invention are clear to a person skilled in the art by the description disclosed herein, and the present invention can be readily reproduced by a person skilled in the art based on the description disclosed herein. It should be noted that the following embodiments of the invention and the specific examples are disclosed solely to exemplify or explain the preferred embodiments of the present invention, and not intended and should not be construed as limiting. Within the aim and the scope of the invention disclosed herein, variations and modifications can be readily made by a person skilled in the art base on the description disclosed herein.

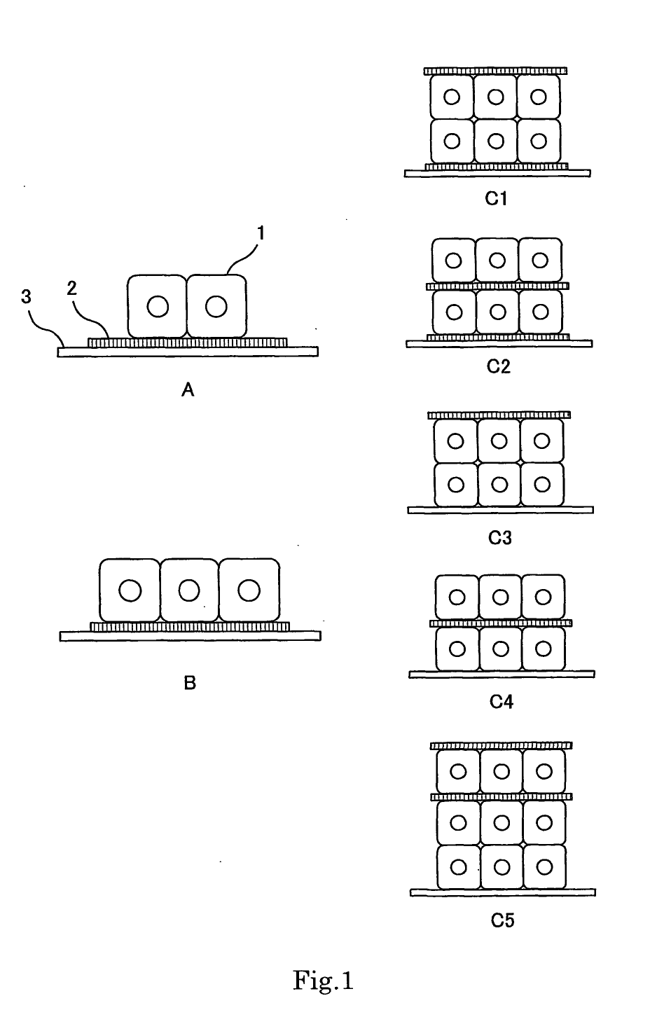

The three-dimensional cell culture method of the present invention is a method to stack the cells having been flat-cultured on a permeable sheet (which is called “cellular sheet” hereafter, including the permeable sheet and the cells), together with the permeable sheet, on other cells having been flat-cultured, thereby to construct a three-dimensional tissue, as illustrated in FIG. 1. The tissue to be constructed can be any tissue, and preferably tissues such as a solid organ that is capable of being sterically reconstructed by the stacking, an epithelial tissue that consists of one to tens layers of cells, and a muscular tissue that has repetitive structures. The origin of the cells is not limited to a particular animal species, but human or swine is favorable in consideration of applications such as transplantation into humans.

First, a tissue is isolated from a living body, from which the cells (1) to be cultured are dissociated, and seeded on a permeable sheet (2) in a culture dish (3) (FIG. 1A). As for the permeable sheet, polycarbonate membranes, collagen membranes, polyester membranes, and any biocompatible membranes made of, for example, polyglycolic acid and polylactic acid, can be used, and biocompatible membranes with properties such as bioabsorbability and/or biodegradability are preferable. The sheet is preferred to be thinner, such as 100 μm or less, more preferably 20 μm or less, and most preferably 10 μm or less. The permeability of the permeable sheet can be as much as the sheet itself can permeate, for example, nutrients, and the sheet can be, for example, a semi-permeable membrane, a permeable membrane, or a microporous membrane which possesses pores with pore-size ranging about 0.01 to about 20 μm. Commercially available sheets usable for the permeable sheet include, but not limited to, the Nuclepore Track-Etched Membrane (WHATMAN, U.K.) and the Permeable Collagen Membrane for Tissue Culture (KOKEN, Japan). The sheet can be treated by a coating, such as collagen coating.

The seeded cells are flat-cultured on the permeable sheet laid in a culture medium (FIG. 1B). The cells can be any cell capable of being flat-cultured, preferably capable of being long-term cultured and maintaining the abilities for three-dimensional tissue construction, such as differentiation and/or proliferation abilities. The cells can be of one type, or more than one types so that the cells of different types can interact with each other, and/or that the cells can construct multiple structures. In one embodiment of the invention, the hepatocytes containing high concentration of small hepatocytes were used.

Cellular sheets are formed in which the cultured cells are attached at high density on the permeable sheet by culturing for an appropriate period. When a microporous sheet is used as the permeable sheet in which pores with an appropriate size are made at appropriate distances, cell colonies may spread depending on anchorages by the pores, thereby the contour of a colony is defined according to the positions of the pores. In this way, the form of colonies can be defined by making the pores at desired positions. Thus, the form of the three-dimensional tissue can be defined by controlling the pore positions, thereby to construct the three-dimensional tissue in a certain form, for example, suitable for transplantation.

The three-dimensional tissue is then constructed by using the cellular sheet formed as above. The cellular sheet made into a monolayered epithelial tissue is organized into the tissue as it is, and thus usable as-is for the transplantation. Tissues with more than one layer, such as solid organs, can be constructed by stacking the cellular sheet on top of other cells having been flat-cultured (FIG. 1 C1 to 5). The stacked layers can be inverted (FIG. 1 C1) or non-inverted (FIG. 1 C2), and the adherent surface between the culture dish and the cells can be without the sheet (FIG. 1 C3, FIG. 1 C4). Not only two but also three or more layers can be stacked up (FIG. 1 C5, for example). Cellular sheets made from the same type of cells can be stacked, as well as cellular sheets made from different types of cells can be stacked.

The cultured cells can be attached to not only one side of the cellular sheet, but also the both sides of the sheet. For example, the commercially available Permeable Collagen Membrane for Tissue Culture (MEN-1, KOKEN) has a base material of the membrane, therefore it can be suspended in a liquid for cell culture so that the cells can be seeded on its both sides of the membrane.

The stacked cellular sheets are cultured further for an appropriate period to organize the cells. As a result, cell adhesions are formed, the cells differentiate, a morphogenesis characteristic to the tissue takes place, and thus the tissue, as a functional cluster of the cells, is constructed.

The three-dimensional hepatic tissue organized as above can be transplanted by itself into human and non-human vertebrates. The site for transplantation can be preferably a liver, as well as other tissues, such as spleen, subcutis, renal subcapsule, testis, and peritoneal cavity.

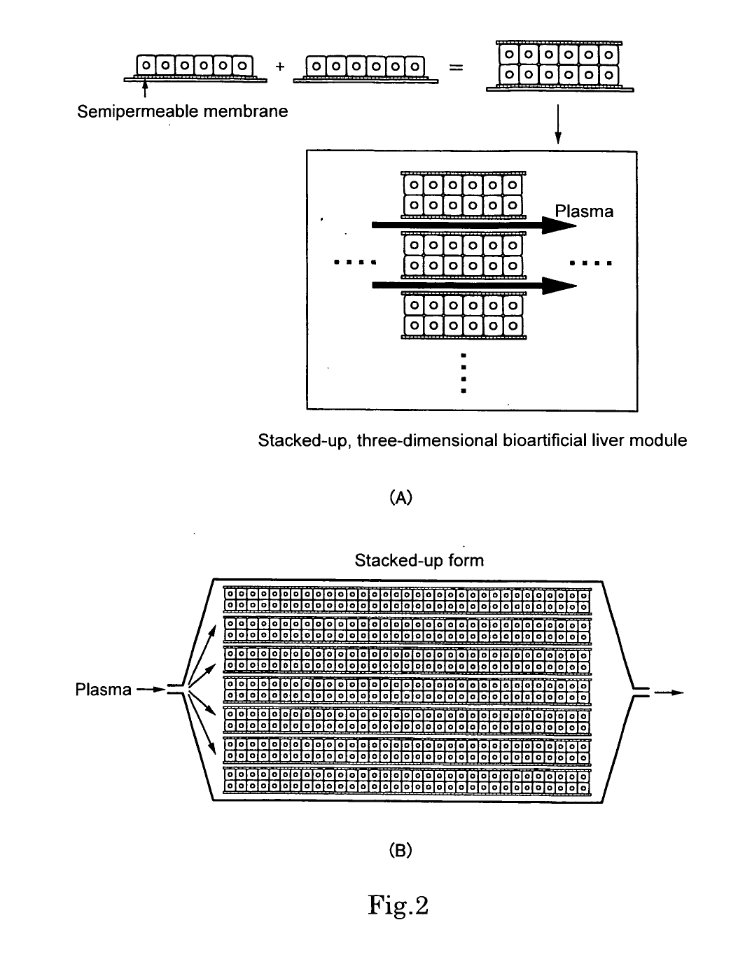

Alternatively, the three-dimensional tissue can be used for a hybrid-type artificial organ. The form of the artificial organ is not limited, but favorable to be a stacked-up form, because a basic construction of the three-dimensional tissue of the present invention is the cellular sheet. As an exemplary artificial organ using the three-dimensional tissue of the present invention, schematic diagrams of a stacked-up, three-dimensional bioartificial liver module is shown in FIG. 2 (A). Small hepatocytes are flat-cultured on permeable membranes, preferably on biocompatible microporous membranes, and then two layers of the cellular sheets are stacked with cellular side of both to adhere to each other, thereby constructing a three-dimensional tissue. The tissues thus constructed with two layers are stacked up with interstices as modules, in a stacked-up artificial liver device, as shown in FIG. 2(B). In the interstices between the tissues, blood or plasma component of blood can be perfused to enable material exchanges with the cells, thereby the device can function as an artificial liver. This artificial liver can function outside of a human body, as well as inside of the human body in an implanted form.

### EXAMPLES

The three-dimensional cell culture method to construct the three-dimensional tissue of the present invention is illustrated hereafter with examples of tissues from a liver. A great number of blood vessels are radiating from the portal vein which runs inside the liver, and the intervascular spaces are filled by bilayers of hepatocytes. Thus, it is understood that the liver is favorable as a most typical embodiment of the present invention.

[Isolation of Small Hepatocytes]

Hepatic cells can be obtained by treating a liver tissue isolated from human or other animals by a solution containing collagenase. Cells were isolated from livers of rats of 8 to 12 weeks of age by a conventional collagenase perfusion method. The cell suspension obtained was sieved through 250 μm and 80 μm meshes to remove undigested tissue debris and other tissue fragments. The suspension was then fractionated by centrifugation at 50×g for 1 min into a heavier fraction which contains mainly parenchymal cells and a supernatant fraction of relatively light cells which contains mainly nonparenchymal cells such as stellate cells, Kupffer cells, and sinusoidal endothelial cells. The small hepatocytes shall be contained in the supernatant fraction at this step. The supernatant was centrifuged at 50×g for 5 min, and the pellet was suspended in a culture medium, and centrifuged again at 50×g for 5 min. The pellet was again suspended in the culture medium and centrifuged at 50×g at 5 min. The pellet thus obtained was further suspended in the culture medium, centrifuged at 150×g for 5 min, and the precipitated cells were suspended in the fresh culture medium. The number of cells in the cell suspension was counted so as to adjust the cell density necessary for the following culture.

[Preparation of Cellular Sheets]

The isolated small hepatocytes are cultured on a collagen-coated microporous polycarbonate membrane (Nuclepore Track-Etched Membrane, WHATMAN). Specifically, 2 ml of the cells adjusted at the density of 3×10⁵ cells/ml were seeded in a 35 mm culture dish containing the microporous polycarbonate membrane which had been coated by the collagen derived from rat tail. As for the culture medium, Dulbecco's modified Eagle's medium supplemented with 10% fetal bovine serum, 0.1 μM dexamethasone, 0.5 mg/l insulin, 10 mM nicotinamide, 1 mM ascorbic acid diphosphate, antibiotics, 10 μg/l epidermal growth factor (EGF), and other supplements generally used for cell culture media was used for the culture at 37° C. After day 4, the culture was supplemented with 1% DMSO. The media were exchanged typically every two days.

[Construction of Three-Dimensional Tissues]

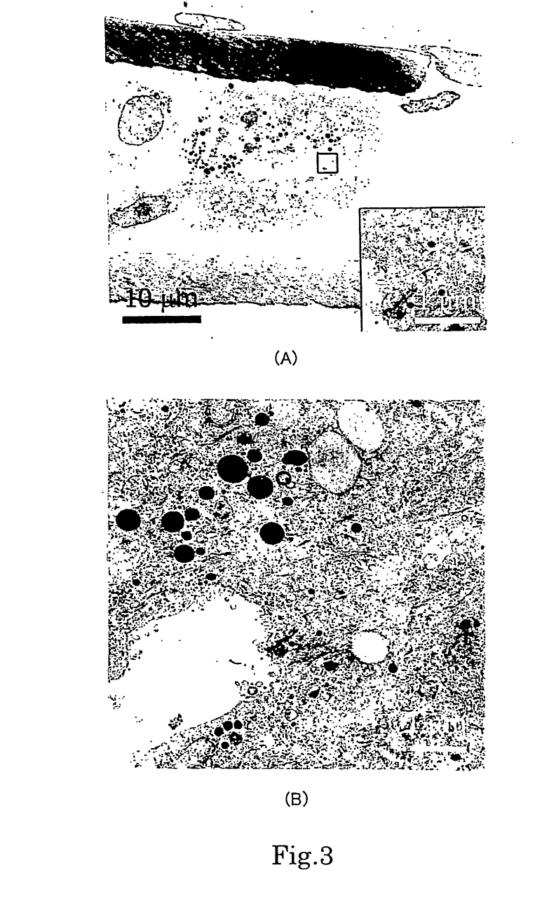

On culture day 30, the cellular sheets were stacked as illustrated in FIG. 1C1, and cultured for several days further in the same condition. The cells were then fixed by 2.5% glutaraldehyde in 0.1 M cacodylate buffer, postfixed, dehydrated, and resin-embedded and perpendicular ultrathin sections were made. Observation of the vertical sections of the stacked-up cellular sheets by a transmission electron microscope revealed intercellular adhesion structures (such as desmosomes and tight junctions) between the upper and lower cells, and thus the upper and lower cells adhered to each other by adhesion molecules (FIG. 3A). Tubular structures between the upper and lower cells were also observed (FIG. 3B), and the structure possessed luminal microvilli, suggesting that they could be the bile canaliculus.

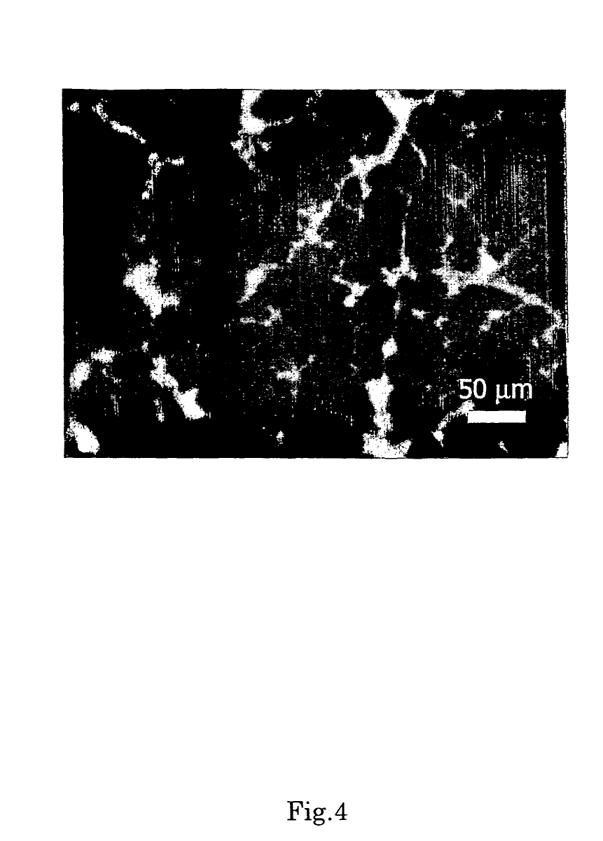

When fluorescein diacetate is administered, hepatocytes can incorporate this substance into cytoplasm, metabolize to fluorescein, a fluorescent substance, and excrete it into the lumen of bile canaliculi. By taking advantage of this ability, it was examined whether the tubular structure observed between the cells of the upper and lower layers in the culture of the present embodiment is a bile canaliculus. The fluorescein diacetate was added to the culture medium at 2.5 μg/ml, and the cells were incubated for 20 min, then washed by warmed medium at 37° C. before fluorescence from the fluorescein was observed by a microscope equipped with a fluorescent detector. As illustrated in FIG. 4, the tubular structure started to be labeled by the fluorescent dye on culture day 3, and thus the tubular structure was shown to be the bile canaliculus.

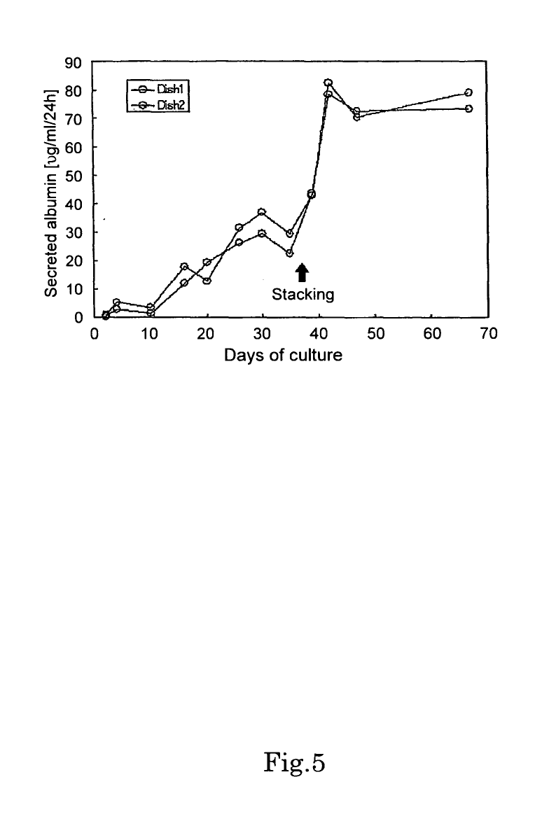

Next, the amount of albumin, a differentiation marker for a hepatocyte, secreted in the medium was measured from the start of the culture and after the stacking of the cells. The culture media 24 hours after the medium exchanges on days 2, 4, 10, 16, 20, 26, 30, 35, 39, 42, 47 and 67 were collected from the same culture dish and freeze-stored. Later, all the frozen samples were thawed, and the amounts of secreted albumin were measured by ELISA (enzyme-linked immunosorbent assay). Whereas the number of cells were doubled by the stacking of the cellular sheets, the level of albumin secretion were quadrupled after the stacking, as illustrated in FIG. 5, indicating that the stacking process accelerated one of the reactions in the cell differentiation.

Taken together, it was demonstrated by the changes in multiple aspects, such as the cell adhesion, the functional morphogenesis, and the cellular differentiation that the cellular sheets were organized by the stacking.

[Defining the Colony Form by Pore Positions]

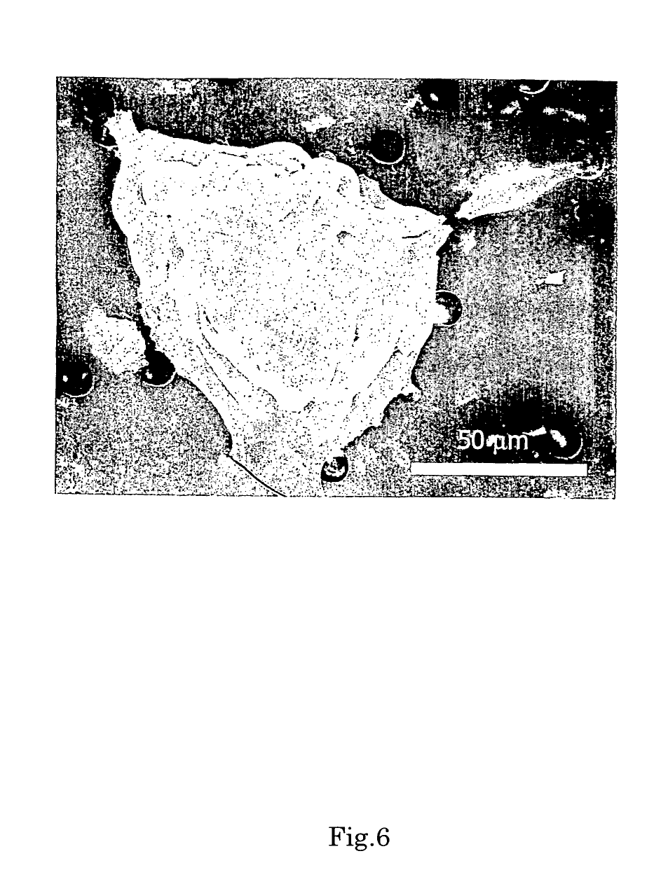

In order to ascertain whether the shape of the colony formed by the small hepatocytes cultured on the polycarbonate membrane matches the positions of the pores, cells on culture day 31 were fixed by 2% glutaraldehyde and 2% osmic acid, and dehydrated by ethanol, to observe the colony form and pore positions by a scanning electron microscope. As illustrated in FIG. 6, peripheral cells of a colony were attached at the positions of pores, and thus the colony form was demonstrated to be defined depending on the pore positions.

[Expression of Hepatocyte Differentiation Markers in the Cells of the Three-Dimensional Tissue]

In order to ascertain whether the cells in the three-dimensional tissue described above are functional as mature hepatocytes, RNA was extracted from cells in the three-dimensional tissue and expressions of hepatocyte differentiation markers were examined.

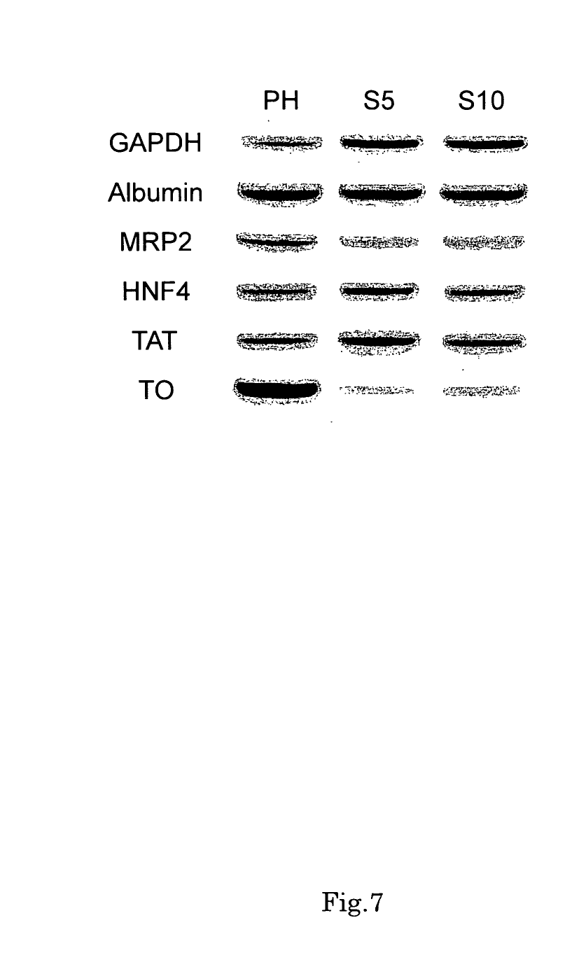

Total RNA was extracted from the cells in the three-dimensional tissue constructed as above by using RNeasy RNA isolation kit (Qiagen), and cDNAs were synthesized by reverse-transcription (at 55° C. for 50 min) of 1 μg of total RNA by using oligo(dT) primer and SuperScript III reverse transcriptase (Invitrogen). cDNAs of albumin, MRP2 (multidrug-resistance associated protein 2), HNF-4 (hepatocyte nuclear factor 4), TAT (tyrosine aminotransferase), TO (tryptophan-2,3-dioxygenase), and GAPDH (glyceraldehyde-3-phosphate-dehydrogenase; used as a control) were amplified by PCR and subjected to agarose gel electrophoresis. The results are shown in FIG. 7.

The PCR reactions were conducted by using Apollo 201 thermal cycler (CLP) with primers shown in Table 1 and Ex Taq (TaKaRa). Reaction conditions for the PCR were as follows: 95° C.×5 min, ->[94° C.×30 seconds, ->Annealing temperature in Table 1×30 seconds, ->72° C.×30 seconds]×Number of cycles in Table 1, ->72° C.×5 min.

TABLE 1

| | | | |
|---|---|---|---|
| | | Annealing | |
| Primer | | temperature | |
| name | Sequence (5′-3′) | (° C.) | Cycles |

| | | | | |
|---|---|---|---|---|
| Albumin | P1 | AAGGCACCCCGATTACTCCG | 56 | 30 |
| | | (Sequence No. 1) | | |
| | P2 | TGCGAAGTCACCCATCACCG | | |
| | | (Sequence No. 2) | | |
| MRP2 | P3 | ACCTTCCACGTAGTGATCCT | 54 | 26 |
| | | (Sequence No. 3) | | |
| | P4 | ACTGTAGGCTCTGGGAAATC | | |
| | | (Sequence No. 4) | | |
| HNF-4 | P5 | TCTACAGAGCATTACCTGGC | 54 | 26 |
| | | (Sequence No. 5) | | |
| | P6 | TGAGGGGAAGATGAAGACGG | | |
| | | (Sequence No. 6) | | |
| TAT | P7 | TACTCAGTTCTGCTGGAGCC | 56 | 26 |
| | | (Sequence No. 7) | | |
| | P8 | GCAAAGTCTCTAGAGAGGCC | | |
| | | (Sequence No. 8) | | |
| TO | P9 | GAAGACGGAGCTCAAACTGG | 56 | 26 |
| | | (Sequence No. 9) | | |
| | P10 | AATAGCGTCTGCTCCTGCTC | | |
| | | (Sequence No. 10) | | |
| GAPDH | P11 | ACCACAGTCCATGCCATCAC | 53 | 30 |
| | | (Sequence No. 11) | | |
| | P12 | TCCACCACCCTGTTGCTGTA | | |
| | | (Sequence No. 12) | | |

As illustrated in FIG. 7, all the hepatocyte differentiation markers examined were expressed in the three-dimensional tissue of the present invention, and thus the cells (hepatic progenitor cells; small hepatocytes) were demonstrated to be functional as mature hepatocytes. This result supports the applicability of the three-dimensional tissues of the present invention to the artificial organs.

### INDUSTRIAL APPLICABILITY

In accordance with the present invention, a cell culture method, a three-dimensional cell culture method to construct a three-dimensional tissue, an artificial organ, and a tissue transplantation method can be provided.

1. A three-dimensional cell culture method comprising constructing a three-dimensional tissue by stacking cells flat-cultured on a permeable sheet on other flat-cultured cells together with said permeable sheet.

2. The three-dimensional cell culture method of claim 1 wherein the cultured cells are originated from any one of a solid organ, an epithelial tissue, or a muscular tissue.

3. The three-dimensional cell culture method of claim 2 wherein the cultured cells are originated from a liver.

4. The three-dimensional cell culture method of claim 3 wherein the cultured cells comprise primarily small hepatocytes.

5. The three-dimensional cell culture method of claim 3 wherein a bile canaliculus is formed in the three-dimensional tissue.

6. A three-dimensional tissue constructed by stacking cells flat-cultured on a permeable sheet on other flat-cultured cells together with said permeable sheet.

7. The three-dimensional tissue of claim 6 wherein the cultured cells are originated from any one of a solid organ, an epithelial tissue, or a muscular tissue.

8. The three-dimensional tissue of claim 7 wherein the cultured cells are originated from a liver.

9. The three-dimensional tissue of claim 8 wherein the cultured cells comprise primarily small hepatocytes.

10. The three-dimensional tissue of claim 8 wherein a bile canaliculus is formed in said three-dimensional tissue.

11. An artificial organ constructed from the three-dimensional tissue of claim 6.

12. A cell culture method of flat-culturing cells on a permeable sheet comprising defining the colony form of the cultured cells by controlling the position of a pore in said permeable sheet.

13. A three-dimensional cell culture method comprising constructing a three-dimensional tissue by stacking cultured cells cultured by the cell culture method of claim 12 on other flat-cultured cells together with the permeable sheet.

14. A tissue transplantation method comprising transplanting the three-dimensional tissue of claim 6 into a living body of a non-human vertebrate.


SEQUENCE LISTING (12 sequences)

SEQ ID NO: 1 - DNA, 20 bp, Artificial Sequence
  Description of Artificial Sequence Synthetic primer
aaggcaccccgattactccg

SEQ ID NO: 2 - DNA, 20 bp, Artificial Sequence
  Description of Artificial Sequence Synthetic primer
tgcgaagtcacccatcaccg

SEQ ID NO: 3 - DNA, 20 bp, Artificial Sequence
  Description of Artificial Sequence Synthetic primer
accttccacgtagtgatcct

SEQ ID NO: 4 - DNA, 20 bp, Artificial Sequence
  Description of Artificial Sequence Synthetic primer
actgtaggctctgggaaatc

SEQ ID NO: 5 - DNA, 20 bp, Artificial Sequence
  Description of Artificial Sequence Synthetic primer
tctacagagcattacctggc

SEQ ID NO: 6 - DNA, 20 bp, Artificial Sequence
  Description of Artificial Sequence Synthetic primer
tgaggggaagatgaagacgg

SEQ ID NO: 7 - DNA, 20 bp, Artificial Sequence
  Description of Artificial Sequence Synthetic primer
tactcagttctgctggagcc

SEQ ID NO: 8 - DNA, 20 bp, Artificial Sequence
  Description of Artificial Sequence Synthetic primer
gcaaagtctctagagaggcc

SEQ ID NO: 9 - DNA, 20 bp, Artificial Sequence
  Description of Artificial Sequence Synthetic primer
gaagacggagctcaaactgg

SEQ ID NO: 10 - DNA, 20 bp, Artificial Sequence
  Description of Artificial Sequence Synthetic primer
aatagcgtctgctcctgctc

SEQ ID NO: 11 - DNA, 20 bp, Artificial Sequence
  Description of Artificial Sequence Synthetic primer
accacagtccatgccatcac

SEQ ID NO: 12 - DNA, 20 bp, Artificial Sequence
  Description of Artificial Sequence Synthetic primer
tccaccaccctgttgctgta